(19) United States
(12) Patent Application Publication
(10) Pub. No.: US 2004/0248120 A1
(43) Pub. Date: Dec. 9, 2004

(54) Detection of specific dinucleotides in dna-samples by fluorescence resonance energy transfer (fret)

(76) Inventor: David Guetig, Berlin (DE)

Correspondence Address: Kriegsman & Kriegsman, 665 Franklin Street, Framingham, MA, 01702, US

(21) Appl. No.: 10/482,339

(22) PCT Filed: Jun. 27, 2002
(86) PCT No.: PCT/DE02/02432

(30) Foreign Application Priority Data
Jun. 27, 2001 (DE) 101-32-211.9

(51) Int. Cl. C12Q 1/68; C12P 19/34

(52) U.S. Cl. 435/6; 435/91.2

(57) ABSTRACT

The invention relates to a method for detecting specific dinucleotides in a DNA-sample. A polymerase-chain reaction (PCR) is carried out by using a) a nucleotide, which is part of the dinucleotide which is to be detected, wherein an adequate quantity thereof is marked by a donor-fluorphore and b) another nucleotide, which is part of the dinucleotide which is to be detected, wherein an adequate quantity thereof is marked with an acceptor-fluorophore. Said method determines or quantifies the presence of the dinucleotide by measuring the dimensions of the fluorescence resonance energy transfer (FRET) between the donor- and acceptor-fluorophore.

### FIELD OF THE INVENTION

This invention concerns the analysis of nucleic acids, particularly the analysis of specific dinucleotides in a specific DNA fragment. This invention further concerns a method for the analysis of methylation patterns in genomic DNA, by providing a means for the detection of CpG dinucleotides which are characteristic for methylated sites of genomic DNA after bisulfite treatment. The method utilizes the incorporation of fluorophores and the detection of fluorescence resonance energy transfer (FRET) of the amplified sample DNA in double-stranded and single-stranded state.

### PRIOR ART

DNA Methylation

The levels of observation that have been studied in molecular biology in recent years have focused on genes, the transcription of these genes into RNA and the translation of the RNA into proteins. Analysis of regulation mechanisms in conjunction with gene control has been extremely limited. Gene regulation, for example, the stage of development of an individual in which a gene is activated or inhibited and the tissue-specific character of this regulation are less well understood. However, this can be correlated with a high degree of probability with the extent and character of the methylation of the gene or genome. It can be meaningfully concluded from this observation that pathogenic genetic anomalies can be detected by means of deviant genetic methylation patterns.

The efforts of the human genome project are focused on the sequencing of the human genome. It is expected that this will bring about significant therapeutic and diagnostic advantages for treating diseases. These efforts, however, were previously incapable of being directed toward a significant aspect of genetic anomalies: the epigenetic factor. It has been shown that the epigenetic regulation of gene transcription causes many anomalies. One of the most significant, previously identified epigenetic mechanisms is the methylation of cytosine. The methylation of cytosine in the 5-position is the only known modification of genomic DNA. Although the precise mechanisms by which DNA methylation influences DNA transcription are unknown, the connection between disease and methylation is well documented. In particular, methylation patterns of CpG islands within regulatory regions of the genome appear to be highly tissue-specific. It follows from this that the erroneous regulation of genes can be predicted by comparison of their methylation pattern with phenotypically “normal” expression patterns. The following examples are cases of disorders which are associated with modified methylation patterns.

Head and neck cancer (Sanchez-Cespedes, M. et al., “Gene promoter hypermethylation in tumours and serum of head and neck cancer patients”, Cancer Res. 2000 Feb. 15; 60(4): 892-5)

Hodgkin's disease (Garcia, J. F. et al.,“Loss of p16 protein expression associated with methylation of the p16INK4A gene is a frequent finding in Hodgkin's disease”, Lab. invest. 1999 December; 79(12): 1453-9)

Stomach cancer (Yanagisawa, Y. et al., “Methylation of the hMLH1 promoter in familial gastric cancer with microsatellite instability”, Int. J. Cancer 2000 Jan. 1; 85(1): 50-3)

Prader-Willi/Angelman syndrome (Zeschnigk et al., “Imprinted segments in the human genome: different DNA methylation patterns in the Prader Willi/Angelman syndrome region as determined by the genomic sequencing method”, Human Mol. Genetics (1997) (6)3, pp 387-395)

ICF syndrome (Tuck-Muller et al., “CMDNA hypomethylation and unusual chromosome instability in cell lines from ICF syndrome patients”, Cytogenet. Call. Genet. 2000; 89(1-2): 121-8)

Dermatofibroma (Chen, T. C. et al., “Dermatofibroma is a clonal proliferative disease”, J. Cutan Pathol. 2000 January; 27(1): 36-9

Hypertonia (Lee, S. D. et al. “Monoclonal endothelial cell proliferation is present in primary but not secondary pulmonary hypertension”, J. Clin. Invest. 1998 March 1, 101(5): 927-34

Autism (Klauck, S. M. et al. “Molecular genetic analysis of the FMR-1 gene in a large collection of autistic patients,” Human Genet. 1997 August; 100(2): 224-9

Fragile X syndrome (Hornstra, I. K. et al., “High resolution methylation analysis of the FMR1 gene trinucleotide repeat region in fragile X syndrome”, Hum. Mol. Genet. 1993 Oct., 2(10): 1659-65)

Huntington's disease (Ferluga, J. et al., “Possible organ and age related epigenetic factors in Huntington's disease and colorectal carcinoma”, Med. hypotheses 1989 May; 29(1); 514)

All of the above documents are incorporated herewith by reference in the [present] disclosure.

Bisulfite Treatment

A relatively new method that is presently the most widely used method for the analysis of DNA relative to 5-methylcytosine is based on the specific reaction of bisulfite with cytosine, which, by subsequent alkaline hydrolysis, is converted to uracil, which corresponds to thymidine in its base-pairing behavior. 5-Methylcytosine remains unchanged, however, under these reaction conditions. Consequently, the original DNA is converted in such a way that methylcytosine, which originally could not be distinguished from cytosine by its hybridization behavior, can now be detected with the use of “standard” molecular biology techniques, such as, for example, by amplification and hybridization or sequencing, as the only remaining cytosine. All of these techniques are based on base pairing, which can now be fully evaluated. With respect to sensitivity, the prior art is defined by a method in which the DNA to be analyzed is enclosed in an agarose matrix, by which means the diffusion and renaturation of the DNA is prevented (bisulfite reacts only with single-stranded DNA), and which replaces all precipitation and purification steps by rapid dialysis (Olek, A.; Oswald, J.; Walter, J. A.; A modified and improved method for bisulphite based cytosine methylation analysis; Nucleic Acid Res., 1966, Dec. 15; 24(24): 5064-6). By use of this method, it is possible to analyze individual cells, which illustrates the potential of this method. At the present time, however, only individual regions of up to a length of approximately 3000 base pairs have been analyzed; an all-encompassing analysis of cells for thousands of possible methylations is not possible. This method, however, cannot reliably analyze very small fragments of small quantities of sample. These are lost despite the protection from diffusion by the matrix.

An overview of other known methods for the detection of 5-methylcytosine can be taken from the following review article: Rein, T.; DePamphilis, M. L.; Zorbas, H.; Nucleic Acids Res., 1998, 26, 2255.

Up to today, apart from a few exceptions (e.g., Zeschnigk M, Lich C, Buiting K, Doerfler W, Horsthemke B. A single-tube PCR test for the diagnosis of Angelman and Prader-Willi syndrome based an allelic methylation differences at the SNRPN locus. Eur. J. Hum. Genet. 1997, March-April; 5(2):94-8), the bisulfite technique is applied only in research. However, short, specific fragments of a known gene have always been amplified after a bisulfite treatment and either completely sequenced (Olek A, Walter J. The pre-implantation ontogeny of the H19 methylation imprint; Nat. Genet., 1997, November; 17(3): 275-6) or individual cytosine positions are detected by a primer extension reaction (Gonzales, M. L.; Jones, P. A. Rapid quantitation of methylation differences at specific sites using methylation-sensitive single nucleotide primer extension (Ms-SNuPE). Nucleic Acids Res., 1997, Jun. 15; 25(12): 2529-31, WO Patent 95-00669) or by enzymatic digestion (Xion, Z, Laird, P W. COBRA: A sensitive and quantitative DNA methylation assay; Nucleic Acids Res., 1997, Jun. 15; 25(12): 2532-4). In addition, detection by hybridization has also been described (Olek et al., WO 99-28498).

Other publications, which are concerned with the use of the bisulfite technique for methylation detection in individual genes: Grigg, G.; Clark, S.; Sequencing 5-methylcytosine residues in genomic DNA, Bioessays, 1994, June; 16(6): 431-6, 431; Zeschnigk, M., Schmitz, B.; Dittrich, B.; Buiting, K.; Horsthemke, B. Doerfler, W.; Imprinted segments in the human genome: different DNA methylation patterns in the Prader-Willi/Angelman syndrome region as determined by the genomic sequencing method; Hum. Mol. Genet., 1997, March; 6(3): 387-95; Feil, R.; Cahrlton, J.; Bird, A. P.; Walter, J.; Reik, W.; Methylation analysis on individual chromosomes: improved protocol for bisulphite genomic sequencing; Nucleic Acids Res., 1994, February 25; 22(4): 695-6; Martin, V.; Ribieras, S.; Song-Wang, X; Rio, M. C.; Dante, R.; Genomic sequencing indicates a correlation between DNA hypomethylation in the 5′ region of the pS2 gene and its expression in human breast cancer cell lines; Gene, 1995, May 19; 157(1-2): 261-4; WO 97/46705, WO 95/15373 and WO 95/45560.

Fluorescence Resonance Energy Transfer (FRET)

Fluorescence resonance energy transfer (FRET) is an interaction between two molecules, in which the excited state of one molecule (the donor) transfers energy to the other molecule (the acceptor). The donor molecule is a fluorophore while the acceptor molecule may or may not be one also. The energy transfer occurs without the emission of photons and is based on the dipole-dipole interaction between the two molecules. Molecules, which are generally used in FRET include fluorescein, N, N, N′, N′-tetramethyl-6-carboxyrhodamine (TAMRA), 6-carboxy-X-rhodamine (ROX), 4-(4′-dimethylaminophenylazo)benzoic acid (DABCYL) and 5-(2′-aminoethylamino)-1-naphthalenesulfonic acid (EDANS).

Standard conditions for FRET include the following:

Close proximity between the donor and acceptor molecules (typically, 10-100×10⁻¹⁰ m).

The emission spectrum of the donor molecule must overlap with the absorption spectrum of the acceptor molecule.

The orientations of the transition dipoles of donor and acceptor molecules must be approximately parallel.

The extent of the energy transfer is dependent on the distance between the two molecules and the overlapping of donor and acceptor spectra. It can be described by the following equation:

kt(r)=tD−1·(R0/r)6

wherein r is the distance between donor and acceptor, tD is the lifetime of the donor in the absence of energy transfer and R0 is designated the Förster distance.

The efficiency of energy transfer (for a single donor-acceptor pair) is given by:

E=R06/(R06+r6)

Förster distances are usually in a range of 30−60×10⁻¹⁰ m. Thus, FRET can be used as a highly sensitive method for the measurement of microscopic distances, which is particularly useful in the field of molecular biology, where it has been used in a number of methods. It has been used in the investigation of protein structure, organization, distribution, conformation and interaction as well as in the investigation of cell membranes and immunoassay. FRET has also been used in a number of methods for the analysis of nucleic acids. These include the structure and conformation analyses of nucleic acids, hybridization, PCR, sequencing and primer extension assay.

Enzymatic Amplification

PCR is a generally used technique, which has been described, for example, in U.S. Pat. Nos. 4,683,195; 4,683,202 and 4,800,159. In short, it is the amplification of a nucleic acid sequence by repetitive cycles of annealing and extension of a primer on single-stranded nucleic acids, followed by the denaturing of the resulting double-stranded molecule. PCR (and variations thereof) has many applications and is one of the key technologies, which is contained in most forms of nucleic acid analysis and manipulation. There are various generally used methods for the detection of PCR products, such as gel electrophoresis and the use of labeled primer oligonucleotides and nucleoside triphosphates. The use of fluorescently labeled nucleotides and oligomers for nucleic acid analysis for PCR is also known.

Genomic DNA for further amplification is obtained from DNA of cells, tissues or other test samples by application of standard methods. This standard methodology is found in references such as Fritsch and Maniatis, eds., Molecular Cloning: A Laboratory Manual, 1989.

Real-time PCR

Real-time PCR monitoring with the use of fluorescence has been described in various ways. As the first way, the binding of dyes with specific fluorescence such as ethidium bromide to double-stranded DNA makes possible monitoring the accumulation of PCR product by correlation with increasing fluorescence. A second detection method, polymerase-supported exonuclease cleavage, makes use of the 5′-exonuclease activity of polymerases such as Taq. An oligonucleotide probe, which is complementary to the PCR product, yet different from the PCR primer, is labeled with a FRET pair, so that the donor molecule is quenched by an acceptor molecule. During the PCR amplification, the 5′-exonuclease begins to digest the probe and separates the FRET pair, which leads to increasing fluorescence. A modification of this technology uses a nucleic acid in which the FRET pair is quenched internally, for example, due to the fact that it possesses a hairpin conformation. By hybridization to a sequence of interest, the FRET pair is separated and the donor molecule emits fluorescence. This technology can be used, for example, for the analysis of SNPs.

An alternative technology is based on the use of two species of hybridization probes, each labeled with one component of the FRET pair. A fluorescence signal is emitted by hybridization of both probes to the target sequence at suitable distance. This technology can also be used for the detection of SNPs.

A principal advantage of the use of such FRET-based PCR technologies is that the reaction can be monitored in a closed-tube reaction, which is suitable for large and medium throughput, and thus reduces the probability of contamination.

### DESCRIPTION OF THE INVENTION

The invention describes a method for the detection of the presence of a specific nucleotide in a DNA fragment, with the use of fluorescence resonance energy transfer (FRET). This can be used in order to obtain information on sequence properties of a sample DNA fragment. For example, a point mutation could be detected in a fragment, if a dinucleotide is present in its sequence as a result of its mutation, which is not present in the wild type. This detection will then function optimally when the dinucleotide formed by the mutation is very rare or is absent in the wild type. Generally, the method will detect modified base sequences, particularly dinucleotides. This limits the application of this method for the detection of mutations, since it is very seldom the case that, in fact, a very short amplificate does not contain a specific dinucleotide or, for example, contains it only once. Therefore, the presented method for the detection of dinucleotides will not be applicable in many cases for the determination of sequence characteristics of genomic DNA samples.

However, the situation is entirely different for genomic DNA which has been treated with bisulfite. As has been mentioned above, bisulfite leads to a selective deamination of cytosine and leaves 5-methylcytosine basically unchanged. The methylation of cytosine occurs almost exclusively in connection with the sequence 5′-CG-3′. Therefore, specific dinucleotides, which contain C, are no longer found in one strand, but they may occur in the complementary strand, which is formed by amplification of bisulfite-treated DNA. The situation is particularly unique for 5′-CG-3′ dinucleotides. These only occur in both strands when the cytosine of the respective CG dinucleotide was methylated and remains unchanged during the bisulfite-supported deamination. Therefore, if a CG dinucleotide exists in an amplificate, and it is presumed that CGs are not contained in the primers, this is a clear indication of the presence of a methylated cytosine in the corresponding genomic DNA sample.

This invention provides a very sensitive method, with a low background signal, for making these dinucleotides visible. As mentioned above, although the detection of CG nucleotides is a preferred application, all other dinucleotides can also be essentially detected. It should be mentioned, however, that the invention cannot essentially distinguish, for example, between GC and CG dinucleotides. However, in bisulfite-treated DNA, for example, GC dinucleotides only occur in the context GCG, since C can still be found only in the context CG. Thus, the presence of GC automatically also demonstrates the presence of CG, so that this aspect does not play a role in the preferred method. Therefore, the detection of dinucleotides will always be described below, even if the distinction of the inverse dinucleotide is not possible.

This method for the detection of specific dinucleotides (or base sequences in general) in a DNA sample is characterized by an amplification step with the use of a polymerase chain reaction (PCR), containing: a) a nucleotide which is part of the dinucleotide to be detected (or base sequence in general), of which a suitable quantity is labeled with a donor fluorophore, and b) another nucleotide which is part of the dinucleotide to be detected (or base sequence in general), of which a suitable quantity is labeled with an acceptor fluorescence. This method is also characterized by the fact that the presence of the dinucleotide is determined by the extent of the fluorescence resonance energy transfer (FRET) between donor and acceptor fluorophores. This means that if the specific dinucleotide to be analyzed is formed, labeled fluorophores are only incorporated in its direct vicinity in the PCR product. For example, if the presence of a CG dinucleotide is to be detected in the PCR product, a fraction of the C nucleotides in the PCR reaction can be labeled with the fluorescent donor and a fraction of the G nucleotides can be labeled with the acceptor fluorophore, or vice versa. FRET can be observed only if a C and G are found close together in the PCR product. In this way, CG dinucleotides can be identified. In contrast, if a TG [di]nucleotide is present, FRET cannot be observed. Therefore, this method can be applied directly in order to observe DNA methylation. This will become apparent from the drawings and their descriptions.

In a preferred embodiment of the invention, a real-time monitoring of the FRET signal is conducted during the PCR. In this way, the progress of the PCR can be investigated. In another preferred embodiment of the invention, the dinucleotide to be detected is self-complementary. For example, this is the case for CG dinucleotides.

In another preferred embodiment of the invention, the dinucleotide to be detected occurs only once in the PCR product. As outlined above, it is very helpful if the presence of the FRET signal is used directly in order to draw conclusions on the sequence characteristics of the DNA sample. For example, if only one CG is present in the PCR product of the bisulfite-treated DNA sample, it can be directly concluded that a methylated cytosine was contained at a specific position in the genomic DNA sample.

However, it is also possible and preferred that the dinucleotide occurs several times in the PCR product and the average quantity of dinucleotides is determined in the PCR product. In this case, for example, the FRET signal is quantified and again, for example, in the case of CG dinucleotides, its quantity in the PCR fragment will be essentially proportional to the observed FRET signal. This can be used to determine the degree of cytosine methylation in a larger DNA fragment.

In another preferred embodiment of the invention, the generation of PCR product is determined by the increase of emitted fluorescence in successive annealing phases, whereas the presence of the dinucleotide to be detected is determined in successive denaturing phases.

Preferably, the sample is illuminated with light of suitable wavelength during the denaturing and the fluorescence is observed as a function of the naturation state of the sample.

The know-how of the invention also lies in the interpretation of a FRET signal in stages in which the sample has double-stranded conformation, as an indicator for a successful amplification reaction and of the FRET signal of the same sample in denatured state, in order to obtain knowledge on the content of CpG dinucleotides in the sample. This is possible, since a FRET pair, formed by a C and a G, which forms a dinucleotide, is independent of the naturation state of the sample, whereas a FRET pair, which is formed by C and G during a Watson-Crick binding, is only present in the double-stranded conformation of the sample, as is illustrated in the figures.

In a preferred embodiment of the invention, prior to the PCR, either essentially all cytosines in the DNA sample are selectively deaminated, but the 5-methylcytosines remain essentially unchanged or all 5-methylcytosines are essentially deaminated, but the cytosines remain essentially unchanged. Cytosine-guanine (CpG) dinucleotides are detected and permit conclusions on the methylation state of the cytosines in these CpG dinucleotides in this DNA sample. This deamination is preferably conducted with the use of a bisulfite reagent.

Preferably, the sample DNA is amplified by selected PCR primers, only if a specific methylation state [is present] at a specific site in the sample DNA, whose sequence context is essentially complementary to one or more of the named selected PCR primers. This can be done with the use of primer annealing selectivity opposite bisulfite-treated DNA, which in a specific position contains either TG or CG, depending on the methylation state in the genomic DNA. Primers can be constructed for both cases. One primer could contain a G at its 3′ end, for which reason it would only bind to a DNA which contains a C at the corresponding position and thus this primer will amplify only or preferably methylated DNA, since the C indicates a methylation in this position after bisulfite treatment. This method is known as MSP, methylation-sensitive PCR.

In another preferred embodiment of the invention, the sample DNA is amplified, only if a specific methylation state is present at a specific site in the sample DNA, whose sequence context is essentially complementary to one or more oligonucleotides or PNA oligomers, which are additionally used in the PCR reaction. Depending on the methylation state of the DNA prior to the bisulfite conversion, these oligonucleotides or PNA oligomers [can also] bind selectively to the template DNA and prevent its amplification.

Preferably, the pairs of donor and acceptor fluorophores are selected from the group consisting of fluorescein/rhodamine, phycoerythrin/Cy7, fluorescein/Cy5, fuorescein/Cy5.5, fluorescein/LC red 640 and fluorescein/LC red 705.

In another preferred variant of the invention, the sample DNA is cleaved with restriction endonucleases prior to the deamination treatment (for example, bisulfite).

A method is also preferred, wherein the enzymatic amplification of the chemically treated DNA is performed such that only one strand of the DNA sample is amplified.

Preferably, the DNA sample is obtained from mammalian sources, e.g., cell lines, blood, sputum, fecal matter, urine, cerebrospinal fluid, tissue embedded in paraffin, for example, tissue from eyes, intestines, kidneys, brain, heart, prostate, lungs, breast or liver, histological sections and all possible combinations.

In another preferred embodiment of the invention, a primer of the PCR reaction is bound to a solid surface. This makes it possible to conduct the amplifications on this surface. The complementary strand can be removed after the amplification and only the dinucleotides in the remaining strand, which is bound to the surface, are analyzed. This is particularly advantageous if the dinucleotide occurs only in one strand and not in the other strand, since the quantity of dinucleotides can be determined independently for both strands. Also, several different PCR reactions can be conducted on one surface, if several different primers are attached to it, in such a way that the position of the primers on the surface correlates with their sequence, so that the evaluation of results is possible.

The surface composition of the named solid phase preferably is comprised of silicon, glass, polystyrene, aluminum, steel, iron, copper, nickel, silver, or gold.

Another preferred embodiment of the present invention is a diagnostic kit for the detection of the methylation of cytosine bases in genomic DNA samples, which comprises reagents for the selective deamination of cytosine bases in genomic DNA, one or more primers and fluorescently labeled dinucleotides for the amplification step and optionally directions or instructions for one of the methods according to one of the preceding claims [sic]. This kit may also consist of several additional articles.

As an example, the components of the named kit could include containers in sufficiently large quantity for the following, in order to conduct the method:

1) Reagents for the bisulfite conversion of the sample DNA.

2) Reagents for the amplification of the converted samples and the incorporation of fluorophore-labeled nucleotides, comprising:

a) Nucleic acid primers

b) Suitable mixture of unlabeled and fluorophore-labeled nucleotides

c) DNA polymerase, which is suitable to incorporate fluorophore-labeled nucleotides

d) Instructions for use

The term “Instructions for use” should cover a comprehensible printed document in order to describe the reagent concentrations for the assay method, parameters such as relative quantities of the reagents to be introduced together, reaction times for reagent/sample mixtures, temperature, buffer conditions and the like.

The steps of the preferred embodiments will be described in detail in the following.

DNA Isolation

First, the genomic DNA sample must be isolated from tissues or cellular sources. In mammals, preferably humans, the DNA sample can be taken from any tissue from which it is assumed that the target region is expressed in the genome, and, for example, from cell lines, blood, sputum, fecal matter, urine, cerebrospinal fluid, tissue embedded in paraffin, for example, tissue from intestine, kidney, brain, heart, prostate, lung, breast or liver, histological sections, but is not limited to these. The extraction can be produced with means familiar to the person skilled in the art, including the use of detergent lysates, ultrasound and vortexing with glass beads. When the nucleic acids have been extracted, the genomic, double-stranded DNA will be used for the analysis.

DNA Restriction

In a preferred embodiment, the DNA can be cleaved prior to the chemical treatment, and this can be done by all means known in the prior art, particularly with restriction endonucleases. Named nucleases can contain cytosine in the 5′-CpG-3′ context in their recognition sequence, so that the DNA is cleaved only if the cytosines are present in the recognition sequence in unmethylated form.

Bisulfite Treatment

The sample DNA is then chemically treated, in order to convert the methylated cytosine bases to uracil. This chemical modification can result, for example, by means of a bisulfite solution, but it is not limited thereto. This chemical conversion can occur in any format known in the prior art. This includes, but is not limited to, modification within agarose gels or in denaturing solvents.

In the case when the chemical modification results as a bisulfite treatment of the DNA, the following steps can be added.

The double-stranded DNA must be denatured. This can be performed as a heat denaturation, which is performed at various temperatures. For high-molecular DNA, the denaturing temperature is usually higher than 90° C. However, the analysis of smaller fragments, which do not require such high denaturing temperatures, may also be conducted. In addition, the complementarity between the strands decreases, as the reaction proceeds and the cytosine residues are converted to uracil. Therefore, a cyclic reaction protocol may include different denaturing temperatures.

The bisulfite conversion additionally comprises two important steps: the sulfonation of the cytosine and the subsequent deamination. The reaction equilibria are found on the correct side at two different temperatures for each step of the reaction. If the reaction kinetics are considered, it is preferred that the reaction occurs under cyclic conditions, with alternating temperatures. The temperatures and reaction times at which each step is conducted can be varied according to the specific requirements in the individual case. However, a preferred variant of the method comprises a temperature change from 4° C. (10 minutes) to 50° C. (20 minutes). This type of bisulfite treatment is prior art in reference to WO 99/28498.

This chemical conversion can occur in any form known in the prior art. This includes, but is not limited to, modification within agarose gels, in denaturing solvents, or within capillaries. The bisulfite conversion within agarose gels is prior art and was described by Olek et al., Nucl. Acids Res. 1996, 24, 5064-5066. The DNA fragment is embedded in agarose gel, and the conversion of cytosine to uracil occurs by means of hydrogen sulfite and a radical trap. The DNA can then be amplified without the necessity for additional purification steps.

In another preferred embodiment, the DNA conversion can occur without the agarose matrix. The DNA can be incubated with hydrogen sulfite and a radical trap at elevated temperatures. This reaction occurs in an organic, denaturing solvent. Examples of denaturing solvents include, but are not limited to, polyethylene glycol dialkyl [sic] polyethylene glycol dialkyl ether, dioxane and substituted derivatives, urea or derivatives, acetonitrile, primary alcohols, secondary alcohols, tertiary alcohols, DMSO or THF.

In another embodiment, the DNA sample is transferred into a capillary, which can be heated and which is permeable to small molecules, prior to the chemical treatment. The reaction steps of the chemical modification can then be conducted by means of addition and removal of reagents via connected capillaries in capillary tubes.

Following the chemical treatment, the two DNA strands could no longer be complementary.

Amplification and incorporation of labeled nucleotides

Fractions of the thus-treated genomic DNA are then amplified enzymatically with the use of oligonucleotide primers. The length and configuration of these primers can be specific to the region of the genome to be analyzed. A large selection of primers is suitable as such for the use of this technique. This primer configuration is prior art.

A suitable fraction of C and G nucleotides, which are introduced into the amplification reaction, are labeled in such a way that a C and a G can form a FRET pair, if they are close to one another. Fluorophore pairs which are suitable for labeling nucleotides so that they are enabled to form FRET pairs, are familiar to the person skilled in the art and include, but are not limited to, fluorescein/rhodamine, phycoerythrin/Cy7, fluorescein/Cy5, fluorescein/Cy5.5, fluorescein/LC red 640 and fluorescein/LC red 705. The coupling of these fluorophores to the nucleotides is prior art.

In a preferred embodiment of this invention, the sample is irradiated with light of a suitable wavelength during the amplification reaction and the fluorescence is recorded as a function of the naturation state of the sample.

The particular feature of the invention lies in the interpretation of a FRET signal in phases in which the sample has double-stranded conformation, as an indicator for a successful amplification reaction, and of the FRET signal of the same sample in denatured state, in order to obtain knowledge on the content of CpG dinucleotides in the sample. This is possible, since a FRET pair, which is formed by a C and a G, which form a dinucleotide, is independent of the naturation state of the sample, whereas a FRET pair, which is formed by a C and G in the scope of Watson-Crick binding, is only present in the double-stranded conformation, which is illustrated by the figures.

Solid-phase Assay

In another preferred embodiment, the primers can be immobilized on a surface. The surface or solid phase can be, for example, a bead, a microplate well or a DNA chip, but is not limited to these. In another preferred embodiment, other reaction participants, such as polymerases, can also be bound to the surface. In such an embodiment, all reagents can be localized in a microplate well, so that the assay can be conducted simply by addition of a suitable buffer and the bisulfite-treated DNA sample.

Subsequent Data Processing

It is assumed that this method is used for the analysis of genomic DNA samples with high throughput. Therefore, the invention also comprises the analysis of data with the use of a computer system. In a preferred embodiment, this system can comprise one or more databases. In another preferred embodiment, this system can comprise one or more “learning algorithms”. Other means for evaluating assay results in the high throughput range are prior art.

### EXAMPLE

For FRET detection, it is necessary to incorporate two different labels in the amplification. This is done preferably by use of a labeled dCTP and a labeled dGTP in a PCR reaction, in order to detect CG dinucleotides on bisulfite-treated DNA and thus roughly the methylation existing in the underlying genomic sample.

HotStar polymerase and the following protocol were used for the PCR:

Thermocycler program:

1. Segment: initial denaturing 15 minutes at 95° C.

2. Segment: 40 cycles of

95° C. for 45 sec

52° C. for 45 sec

72° C. for 45 sec

3. Final synthesis phase 10 minutes at 72° C.

Fluorescein and Cy5 were selected as the donor-acceptor pair. These fluorophores are suitable for use on a LightCycler.

FRET was measured both for the single strand, i.e., between labels on one strand, and FRET between two hybridized strands. A FRET between hybridized strands was only very weak, so that it does not significantly influence the evaluation. The following PCR experimental conditions applied to the simultaneous incorporation of fluorescein-G and Cy5 [-C] labels for the detection of a FRET in the single strand in a PCR reaction:

Fluo-dGTP 0.050 mM

Cy5-dCTP 0.050 mM

Taq 0.1 U/μl

dNTP 0.1 mM (each)

Primer 1 0.5 pmol/μl

Primer 2 0.5 pmol/μl

BSA 0.25 mg/ml

Buffer 1×1.5 mM

DNA 0.4 ng/μl

A 153-bp fragment of the bisulfite-treated human GSTPI gene was selected as an example. The following primers were used for the amplification:

| | | |
|---|---|---|
| Primer 1: | GTTTT (C/T) GTTATTAGTGAGT | (SEQ. ID: 1) |
| Primer 2: | TCCTAAATCCCCTAAACC | (SEQ. ID: 2) |

For detection of the FRET between two strands and the FRET between different labels within one strand, first of all, heteroduplexes were prepared from amplificates which were each provided with only one of the labels, and secondly, amplificates were prepared according to the above protocol, in which both labels were incorporated in one strand, with labeled dCTP und dGTP.

The PCR reaction was analyzed by using an agarose gel according to standard methods. The length of the PCR products was investigated by fluorescence detection on a polyacrylamide gel (ALF-Express Instrument, Amersham Pharmacia) and fragment analysis by means of capillary gel electrophoresis (ABI Prism).

The application of the ALF standard protocol to the monitoring of the incorporation of the respective labels is shown in FIG. 3 and FIG. 4. The FRET between the labels was determined on a LightCycler Instrument (Roche Applied Science).

The FRET between fluorescein and Cy5 now enables conclusions on the number of CpGs in a specific volume element in the sample. The intensity of the FRET is dependent on the rate of incorporation of the labeled nucleotides, the average methylation degree, the number of CpGs in the sample and the concentration of the fragments. The incorporation rate can be determined by fragment analysis as in FIG. 4 by means of an internal standard (on the primer), [if] the concentration of the fragments as well as the number of CpGs are known. Thus the average number of CpGs can be determined, and from this, the methylation degree can be determined without doubt; verification is made by known templates with 0 and 100% methylation.

The intensity of the FRET is also dependent on the distance between the incorporated dyes. Especially, if one would like to determine specific dinucleotides, knowing the dependence of the FRET on the distance between the dyes is indispensable . This can be determined via probe oligonucleotides in a LightCycler experiment such as the following, here by a melting curve: If hybridization probes were used for the detection with a gap of 4 bases between the probes:

1. Anchoring probe with fluorescein label on the 3′-end (*):

5′-GTTTAGAGTTTTTAGTATGGGGTTAATT_*

2. Methylation-specific probe with Cy5 label on the 5′-end (*):

*_5′-GTATTAGGTTTGGGTTTTTGGT

The FRET detection of the probes takes place as a melting curve determination in a LightCycler (FIG. 5). Initially higher fluorescence values are achieved by the FRET. Due to the melting of the DNA, the strands are separated to such an extent that the energy transfer between the fluorescein and Cy5 labels is no longer possible and the fluorescence approaches the background signal.

This melting curve analysis for the FRET detection can be conducted in the method according to the invention following the PCR for the determination of the number of FRET pairs in a specific volume element.

From this number of FRET pairs, the concentration of the PCR products and the number of methylatable positions, finally the average methylation degree is calculated with knowledge of the incorporation rate (see above).

### DESCRIPTION OF THE FIGURES

FIG. 1a

Double-stranded PCR fragment of a bisulfite DNA probe, in which a cytosine (C) has remained unchanged, since it was methylated. The Gs and Cs are labeled in each case with a donor fluorophore and an acceptor fluorophore and form FRET pairs, since they are found in close proximity to one another.

FIG. 1b

Double-stranded PCR fragment of a bisulfite DNA probe with the same original sequence as in FIG. 1a, wherein a C was converted to T, since it was not methylated. A FRET pair is still formed by a labeled G and a labeled C on the opposite-lying strands.

FIG. 2a

A PCR fragment of FIG. 1a under denaturing conditions, wherein the individual strands are separated to such an extent that FRET pairs cannot form by Gs and Cs on the opposite-lying strands. A FRET signal can still be detected, since FRET pairs form by Gs and Cs on the individual strands.

FIG. 2b

A PCR fragment of FIG. 1a under denaturing conditions, wherein the individual strands are separated to such an extent that neither a FRET pair is formed by Gs and Cs on the opposite-lying strands, nor a FRET pair is formed on the individual strands, since either only a G or a C is present. Therefore, a FRET signal cannot be detected.

FIG. 3

Detection on ALF Express with standard protocol (only Cy5 detected): Completely methylated, bisulfite-treated and amplified DNA: Peak A: C-rich strand labeled with Cy5; Peak B: G-rich strand labeled with Cy5 (i.e., only the cytosines from CG dinucleotides); Unmethylated, bisulfite-treated and amplified DNA; Peak C: C-rich strand labeled with Cy5; D: G-rich strand, no peak, since no cytosines present.

FIG. 4

Detection on ABI Prism 310 DNA analyzer (only fluorescein detected): Completely methylated, bisulfite-treated and amplified DNA: Peak A: C-rich strand labeled with fluorescein, only visible in methylated state; B: G-rich strand labeled with fluorescein; Unmethylated, bisulfite-treated and amplified DNA; C: C-rich strand, no peak; peak D: G-rich strand labeled with fluorescein.

FIG. 5

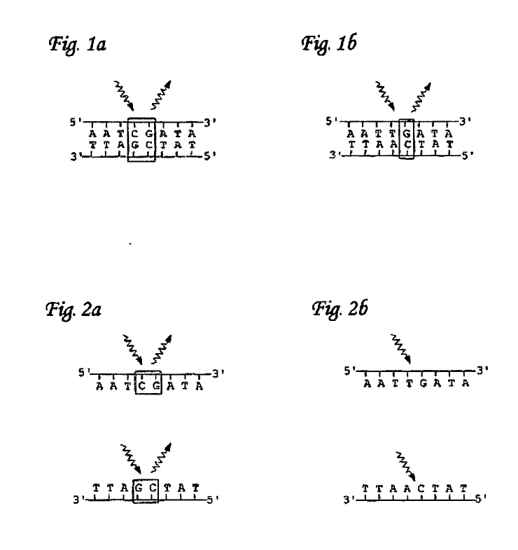

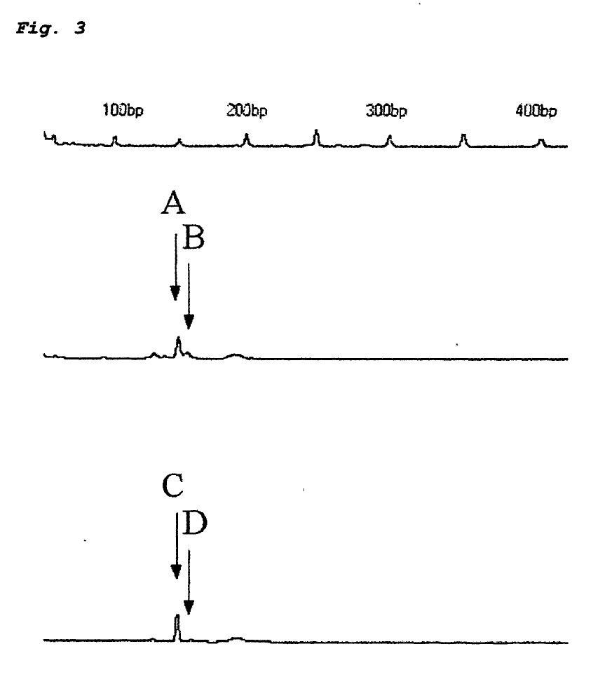

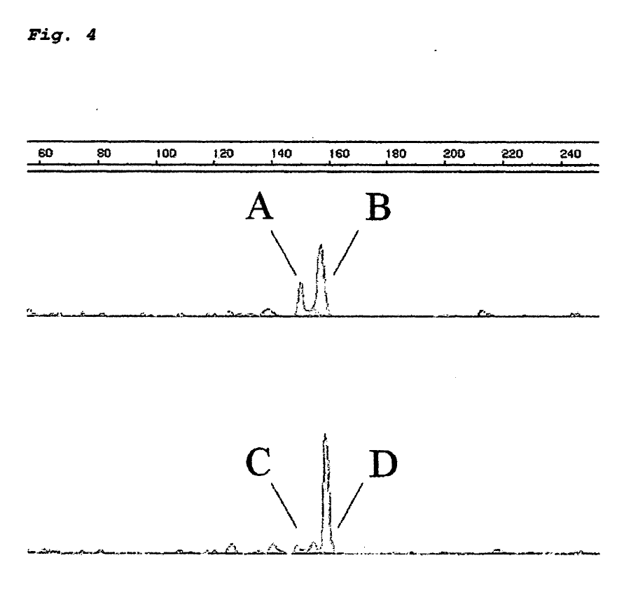

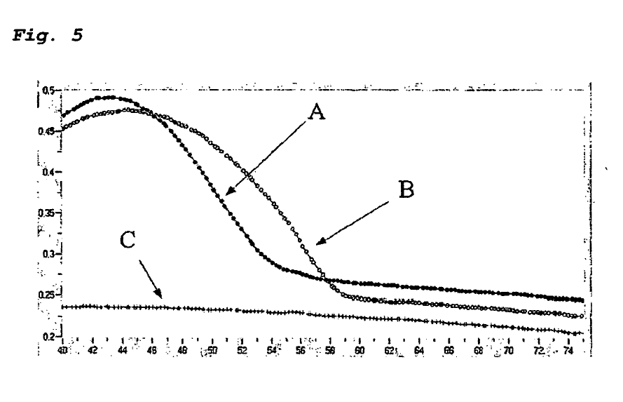

The curves represent the melting point determinations, which detect the FRET between the two detection probes. Curve A shows the melting curve of the methylated, bisulfite-treated DNA; curve B shows the bisulfite-treated, unmethylated DNA; and C shows the background signal. The lower melting point of A occurs due to mismatch.

1. A method for the detection of specific base sequences in a DNA sample is hereby characterized in that a polymerase chain reaction PCR) is conducted with the use of
a) a nucleotide, which is part of the base sequence to be detected, of which a suitable quantity is labeled with a donor fluorophore,
b) another nucleotide, which is part of the base sequence to be detected, of which a suitable quantity is labeled with an acceptor fluorophore,
and is hereby characterized in that the presence of the dinucleotide is determined by the extent of the fluorescence resonance energy transfer (FRET) between donor and acceptor fluorophores.

2. The method according to claim 1, further characterized in that a real-time monitoring of the FRET signal is conducted during the PCR.

3. The method according to claim 1, further characterized in that the dinucleotide to be detected is self-complementary.

4. The method according to claim 1, further characterized in that the dinucleotide to be detected occurs only once in the PCR product.

5. The method according to claim 1, further characterized in that the dinucleotide occurs several times in the PCR product and the average quantity of dinucleotides is determined in the PCR product.

6. The method according to claim 1, further characterized in that the generation of PCR product is observed by the increase of emitted fluorescence in successive annealing phases, whereas the presence of the dinucleotides to be detected is determined in successive denaturing phases.

7. The method according to claim 1, further characterized in that prior to the PCR, either
essentially all cytosines in the DNA sample are selectively deaminated, but the 5-methylcytosines remain essentially unchanged or
all 5-methylcytosines are essentially selectively deaminated in the DNA sample, but the cytosines remain essentially unchanged and
is hereby characterized in that cytosine-guanine (CpG) dinucleotides are predominantly detected, which permits conclusions on the methylation state of the cytosines in these CpG dinucleotides of this DNA sample.

8. The method according to claim 7, further characterized in that the deamination is conducted by a bisulfite reagent.

9. The method according to claim 1, further characterized in that the sample DNA is amplified by selected PCR primers, only if a specific methylation state is present at a specific site in the sample DNA, whose sequence context is essentially complementary to one or more of the selected primers.

10. The method according to claim 1, further characterized in that the sample DNA is amplified by selected PCR primers, only if a specific methylation state is present in the sample DNA, whose sequence context is essentially complementary to one or more oligonucleotides or PNA oligomers, which are additionally used in the PCR reaction.

11. The method according to claim 1, wherein the pairs of donor and acceptor fluorophores are selected from the group consisting of fluorescein/rhodamine, phycoerythrin/Cy7, fluorescein/Cy5, fuorescein/Cy5.5, fluorescein/LC red 640 and fluorescein/LC red 705.

12. The method according to claim 1, wherein the DNA sample is cleaved with restriction endonucleases prior to the deamination treatment.

13. The method according to claim 1, wherein the enzymatic amplification of the chemically treated DNA is of such type that only one strand of the DNA sample is amplified.

14. The method according to claim 1, wherein the DNA sample is isolated from mammalian sources, e.g., cell lines, blood, sputum, fecal matter, urine, cerebrospinal fluid, tissue embedded in paraffin, for example, tissue from eyes, intestines, kidneys, brain, heart, prostate, lungs, breast or liver, histological sections and all possible combinations.

15. The method according to claim 1, further characterized in that a primer is bound to a solid surface in the PCR reaction.

16. The method according to claim 15, wherein the surface composition comprises silicon, glass, polystyrene, aluminum, steel, iron, copper, nickel, silver, or gold.

17. A diagnostic kit for the detection of the methylation of cytosine bases in genomic DNA samples, comprising reagents for the selective deamination of cytosine bases in genomic DNA, one or more fluorescently labeled nucleotides for the amplification step and, optionally, protocols or instructions for a method according to one of the preceding claims.


SEQUENCE LISTING (4 sequences)

SEQ ID NO: 1 - DNA, 19 bp, artificial sequence
  Primer
gttttygttattagtgagt

SEQ ID NO: 2 - DNA, 18 bp, artificial sequence
  Primer
tcctaaatcccctaaacc

SEQ ID NO: 3 - DNA, 28 bp, artificial sequence
  labelled probe
gtttagagtttttagtatggggttaatt

SEQ ID NO: 4 - DNA, 22 bp, artificial sequence
  labelled probe
gtattaggtttgggtttttggt